(19) United States
(12) Patent Application Publication
(10) Pub. No.: US 2024/0239913 A1
(43) Pub. Date: Jul. 18, 2024

(54) COMPOSITIONS AND METHODS FOR TREATING OSTEOARTHRITIS USING A CD14 INHIBITOR

(71) Applicants: The United States Government As Represented By The Department Of Veterans Affairs, Washington, DC (US); THE TRUSTEES OF THE UNIVERSITY OF PENNSYLVANIA, Philadelphia, PA (US); OKLAHOMA MEDICAL RESEARCH FOUNDATION, Oklahoma City, OK (US)

(72) Inventors: CARLA R. SCANZELLO, Philadelphia, PA (US); TIMOTHY GRIFFIN, Oklahoma City, OK (US)

(21) Appl. No.: 18/411,242

(22) Filed: Jan. 12, 2024

Related U.S. Application Data
(60) Provisional application No. 63/479,605, filed on Jan. 12, 2023.
(60) Provisional application No. 63/484,099, filed on Feb. 9, 2023.

(51) Int. Cl. C07K 16/28; A61P 19/02; C07K 14/745; C12N 9/12; A61K 38/00; A61K 39/00

(52) U.S. Cl. CPC C07K 16/2896; A61P 19/02; C07K 14/7455; C12N 9/12; A61K 38/00; A61K 2039/505; C12Y 207/12002

(57) ABSTRACT

Disclosed herein are methods of treating or preventing the development of osteoarthritis after a joint injury by administering a CD14 inhibitor capable of neutralizing or blocking CD14, inhibiting CD14 function, inhibiting CD14 production, or a combination thereof. Also disclosed herein are methods of reducing or ameliorating one or more symptoms of osteoarthritis, reducing inflammation, reducing cartilage degradation, or treating or preventing subchondral bone sclerosis in a subject.

### CROSS REFERENCE TO RELATED APPLICATIONS

This application claims the benefit of U.S. Provisional Application No. 63/479,605, filed Jan. 12, 2023, and U.S. Provisional Application No. 63/484,099, filed Feb. 9, 2023. The content of this earlier filed application is hereby incorporated by reference herein in its entirety.

### STATEMENT REGARDING FEDERALLY FUNDED RESEARCH

This invention was made with government support under grant numbers R01AR075737, P30AR069619, and P30AR073750 awarded by the National Institutes of Health, and I01-BX004912 and I01-BX004882 awarded by United States Department of Veterans Affairs. The government has certain rights in the invention.

### BACKGROUND

Current treatment options for osteoarthritis (OA) include anti-inflammatory agents such as oral non-steroidal anti-inflammatory drugs (NSAIDs) and intra-articular glucocorticoids. These options are limited to ameliorating pain temporarily without impacting disease progression. Moreover, existing anti-inflammatory agents such as the oral NSAIDs increase risk of gastric ulceration and cardiovascular events, can cause renal and hepatic toxicity, and are contraindicated in patients on anti-coagulation therapies. These side effects and toxicities limit their use in many OA patients who tend to be of older age and have medical comorbidities. Inflammation and bone remodeling are strong pathologic correlates of OA pain (Hunter D J, et al. Osteoarthritis Cartilage. 2013; 21(9):1170-8), but recent trials of existing anti-inflammatory and anti-resorptive drugs developed for other forms of arthritis have not been successful in OA (Cohen S B, et al. Arthritis Res Ther. 2011; 13(4):R125; Aitken D, et al. 2018; 26(7):880-7; and Davis A J, et al. PLOS One. 2013;8(9):e72714). For example, trials of individual cytokine blockade (anti-TNF, anti-IL1) and trials of bisphosphonates that target bone resorption have each failed to demonstrate meaningful effects on either joint pain or structural progression. Thus, a need exists for the treatment and management of OA.

### SUMMARY OF THE INVENTION

Disclosed herein are methods of treating osteoarthritis or preventing the development of osteoarthritis after a joint injury in a subject, the methods comprising: administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteoarthritis or preventing the development of osteoarthritis after a joint injury in the subject.

Disclosed herein are methods of reducing or ameliorating one or more symptoms of osteoarthritis in a subject, the methods comprising: administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing or ameliorating one or more symptoms of osteoarthritis in the subject.

Disclosed herein are methods of reducing inflammation in a subject, the method comprising: administering to the subject with osteoarthritis or at risk for developing osteoarthritis after a joint injury, a therapeutically effective amount of a CD14 inhibitor, wherein the therapeutically effective of the CD14 inhibitor amount neutralizes or blocks CD14, inhibits CD14 function, inhibits CD14 production, or a combination thereof, thereby reducing inflammation in the subject.

Disclosed herein are methods of reducing cartilage degradation in a subject, the methods comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing cartilage degradation in the subject.

Disclosed herein are methods of treating or preventing subchondral bone remodeling in a subject, the methods comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing subchondral bone remodeling in the subject.

Disclosed herein are methods of treating or preventing subchondral bone sclerosis in a subject, the methods comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing subchondral bone sclerosis in the subject.

Disclosed herein are methods of treating or preventing osteophytosis in a subject, the methods comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing osteophytosis in the subject.

Disclosed herein are methods of treating or preventing bone marrow lesions in a subject, the methods comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing bone marrow lesions in the subject.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-F show that CD14 deficiency prevents pain-related functional decline, subchondral bone remodeling, and progression of cartilage degeneration in a mouse model of post-traumatic OA. CD14−/− mice and congenic C57BL/6 controls were subjected to destabilization of the medial meniscus model (DMM) surgery (transection of the medial meniscotibial ligament, MMTL), sham surgery (exposure of the MMTL without transection), or left unmanipulated (naïve). Spontaneous behavior impacted by pain was measured using the LABORAS® system (laboratory animal behavior observation registration and analysis system, Metris B.V., Hoofdorp, The Netherlands). WT mice decreased climbing activity from pre-operative levels by 50% at 4 and 8 weeks post-DMM, while CD14−/− mice were protected from this decline (FIGS. 1A-B). Knee joints were harvested at 6 and 19 weeks post-DMM for analysis of tibial subchondral bone mineral density (BMD) by microCT (Scanco, Inc.), followed by cartilage histopathology. BMD (FIGS. 1C-D) increased in WT mice, but remained stable in the CD14-deficient strain. Using the modified OARSI score, early cartilage degeneration was similar in both strains, while at 19 weeks post-DMM, damage was more severe in WT mice indicating less progression in the absence of CD14 (FIGS. 1E-F).

FIGS. 2A-D show intra-articular (IA) injection with a CD14 blocking antibody reduces functional decline after destabilization of the medial meniscus model (DMM) injury in male mice, and does not worsen joint pathology. Effects of IA anti-CD14 mAb treatment on joint pathology and spontaneous activity. FIG. 2A is a schematic of experiment. FIG. 2B shows total OARSI cartilage and osteophyte scores at 4 weeks post-DMM (unop=unoperated side). FIG. 2C shows a comparison of two doses of anti-CD14 on medial tibial cartilage pathology 4 weeks post-DMM. FIG. 2D shows rearing activity, expressed as the change from pre-operative activity, 2 & 4 weeks post-DMM.

FIG. 3 shows an experimental design to assess the effects of intra-articular (IA) injection of a CD14 blocking antibody on pain and structural outcomes in a non-invasive compression injury model that causes anterior cruciate ligament rupture and post-traumatic osteoarthritis (PTOA) tested in 36-week-old female mice fed a high fat diet for 20 weeks.

FIGS. 4A-B show the pre-injury responses across the von Frey filaments. FIG. 4A shows the pre-injury responses across all 7 von Frey filaments. FIG. 4B shows the pre-injury responses across the first 4 von Frey filaments. Von Frey filaments applied in ascending order to left and right hind paws, repeated twice. Analyzing the first four filaments generates an average pre-injury withdrawal response rate of approximately 0.5 or 50%.

FIG. 5 shows that mechanical allodynia increased 2- and 4-weeks post-injury compared to pre-injury. Bars represents the mean±95CI. P-values indicates statistical significance across time. Data points represent individual animal average withdrawal response (%) for the seven filaments.

FIGS. 6A-B show the effects of injury on mechanical allodynia measured by von Frey filament withdrawal response (seven filaments) in mice treated with control or anti-CD14 treatment. No difference was observed at pre-injury (baseline) (FIG. 6A). At 4 weeks post compression injury, the withdrawal response was lower with anti-CD14 treatment. Bars represents the mean±95CI. P-value indicates statistical significance.

FIGS. 7A-B show the averages of the first 4 von Frey filaments. Effects of injury on mechanical allodynia (increased percent von Frey filament withdrawal response from pre-injury) in mice treated with IgG control or anti-CD14 treatment. No difference was observed at pre-injury (baseline) (FIG. 7A). At 4 weeks post compression injury, the withdrawal response was lower with anti-CD14 treatment (FIG. 7B). Bars represents the mean±95CI. P-value indicates statistical significance.

FIG. 8 shows the effects of injury on hindlimb body weight support ratio (uninjured limb:injured limb) in mice pre-injury and post compression injury measured by dynamic weight bearing testing. Bars represents the mean±95CI. P-values indicates statistical significance.

FIG. 9 shows the effects of injury on body weight support by the injured hindlimb in mice pre-injury and post compression injury measured by dynamic weight bearing testing. Bars represents the mean±95CI. P-values indicates statistical significance.

FIG. 10 shows the effects of injury on body weight support by the uninjured contralateral hindlimb in mice pre-injury and post compression injury measured by dynamic weight bearing testing. Anti-CD14 treatment prevented the reduction in contralateral hindlimb bodyweight support at 4-weeks post injury as occurred with control treatment. Bars represents the mean±95CI. P-values indicates statistical significance.

FIG. 11 shows the effects of injury on forelimb body weight support relative to hindlimb body weight support in mice pre-injury and post-compression injury measured by dynamic weight bearing testing. Anti-CD14 treatment reduces the shift in body weight support from hindlimbs to forelimbs 4 weeks after injury compared to control treatment. Bars represents the mean±95CI. Significance*=P-values indicates statistical significance.

FIG. 12 shows the effects of injury on the percent of time spent rearing on the hindlimbs in mice pre-injury and post compression injury measured by dynamic weight bearing testing. Anti-CD14 treatment prevented the reduction in hindlimb rearing behavior in mice at 4 weeks post injury compared to control treatment. Bars represents the mean±95CI. P-values indicates statistical significance.

FIG. 13 shows the effects of injury on pain behavior in mice 2- and 4-weeks post compression injury measured by knee Algometer testing. Maximum Targeted Withdrawal Threshold: 1000 g. P-values indicates statistical significance.

FIGS. 14A-B show the effects of injury on pressure hyperalgesia in mice pre- and 4-weeks post compression injury measured by Algometer testing. Individual responses in the same animal animals pre- and post-injury for control (FIG. 14A) and anti-CD14 antibody (FIG. 14B) treatment on pain behaviors are shown. P-value indicates statistical significance.

FIG. 15 shows the effects of injury on generalized pain behavior in mice 2- and 4-weeks post compression injury measured by Algometer testing in the non-injured contralateral limb. Anti-CD14 treatment prevented the development of generalized hyperalgesia at 4-weeks post compression injury compared to control treatment. P-values indicates statistical significance.

FIGS. 16A-C show the effects of intra-articular (IA) injection of a CD14 blocking antibody on pain and structural outcomes in the destabilization of the medial meniscus model (DMM) of post-traumatic osteoarthritis (PTOA) tested with an 8-week endpoint. FIG. 16A shows an experimental design. FIG. 16B shows that at 8-weeks post DMM, no significant difference was observed in cartilage pathology scoring (OARSI score) in DMM-operated knees between anti-CD14 and control treated groups. FIG. 16C shows that anti-CD14 treatment significantly increased rearing time at 4- and 8-weeks post DMM, compared to control treated mice (p<0.05).

FIGS. 17A-C show anti-CD14 treatment reduces knee joint pathology 4 weeks compared to IgG control following non-invasive knee compression injury that ruptures the anterior cruciate ligament. FIG. 17A shows that anti-CD14 treatment significantly reduced cartilage pathology. FIG. 17B shows that anti-CD14 treatment reduced fibrosis (score averaged for all sites), but did not alter synovial hyperplasia or cellularity. P-values were derived from Mann-Whitney tests (cartilage pathology) or unpaired Student's t-test (synovial pathology) based on residual and variance analyses. Data points show values for individual animals, and boxes represent the 25th to 75th percentiles, horizontal line indicates the median, and whiskers demonstrate maximum and minimum values. FIG. 17C shows representative histology images illustrating improved preservation of articular cartilage along the femur and tibia (arrowheads) in anti-CD14 treated animals. Scale bar=500 μm.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

The use of the word “a” or “an” when used in conjunction with the term “comprising” in the claims and/or the specification may mean “one,” but it is also consistent with the meaning of “one or more,” “at least one,” and “one or more than one.”

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list. The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Treatment” and “treating” refer to administration or application of a therapeutic agent (e.g., a CD14 inhibitor) to a subject or performance of a procedure or modality on a subject for the purpose of obtaining a therapeutic benefit of a disease or health-related condition. For example, a treatment may include administration of a pharmaceutically effective amount of CD14 inhibitor capable of neutralizing or blocking CD14, inhibiting CD14 function, inhibiting CD14 production, or a combination thereof.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition (e.g. osteoarthritis, inflammation, cartilage degradation, or subchondral bone sclerosis). Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be osteoarthritis, inflammation, cartilage degradation, bone marrow lesions, osteophytosis, or subchondral bone sclerosis.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. In some aspects, a subject is a non-human primate. The term does not denote a particular age or sex. Thus, adult, child, adolescent and newborn subjects, as well as fetuses, whether male or female, are intended to be covered.

As used herein, the term “patient” refers to a subject afflicted with a condition, disease or disorder (e.g., osteoarthritis, inflammation, cartilage degradation, bone marrow lesions, osteophytosis, or subchondral bone sclerosis). The term “patient” includes human and veterinary subjects. In some aspects of the disclosed methods, the “patient” has been diagnosed with osteoarthritis, inflammation, cartilage degradation, bone marrow lesions, osteophytosis, or subchondral bone sclerosis. In some aspects of the disclosed methods, the “patient” has been diagnosed with a need for treatment (e.g. treatment for osteoarthritis, inflammation, cartilage degradation, bone marrow lesions, osteophytosis, or subchondral bone sclerosis), such as, for example, prior to the administering step.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Osteoarthritis (OA) is the most prevalent form of arthritis worldwide; affecting an estimated 27 million patients in the U.S., and its socioeconomic impact is estimated at $60 billion per year. Joint injuries such as meniscal tears and ligament injuries are known risk factors for development of OA in the injured joint, and this “post-traumatic” subset accounts for about 12% of OA cases. Currently no therapies are available that prevent OA progression of joint damage and disability, or prevent development of OA after a joint injury. Joint inflammation is linked to severity of pain and progression of disease, but current anti-inflammatory medications are short-acting, do not protect against progression of disease or disability, and have substantial side effects.

Described herein are compositions and methods for blocking or inhibiting the earliest events that trigger inflammation in response to joint tissue damage, by, for example, blockade, inhibiting the function of, inhibiting the production of or neutralization of the molecule CD14 which is involved in triggering inflammation after injury. The methods of treatment disclosed herein are directed to preventing arthritis development after an injury, and treating progression of arthritis and arthritis pain. The methods of treatment disclosed herein are directed to preventing or treating or inhibiting osteoarthritis, inflammation, cartilage degradation, bone marrow lesions, osteophytosis, or subchondral bone sclerosis in a subject. The compositions and methods disclosed herein have distinct advantages over existing anti-inflammatory treatments which are very short-acting and do not prevent arthritis or arthritis progression, including, but not limited to (i) targeting the pathways that initiate inflammation in response to joint damage, thus acting far upstream of available agents, (ii) dampening the effects of multiple redundant TLR ligands and pathways implicated in OA, and thus will have a broader effect than blocking individual TLR ligands, receptors or cytokines, and (iii) the approach disclosed herein will be safer than existing therapies, as clinical studies with an anti-human CD14 blocking antibody, used for other indications, have suggested significant safety, and (iv) will be as or more effective at alleviating symptoms of osteoarthritis, inflammation, cartilage degradation, bone marrow lesions, osteophytosis, or subchondral bone sclerosis than currently available injectable agents such as intra-articular corticosteroids or viscosupplementation agents. Expanding medical approaches for OA and joint injuries can improve quality of life, enhance rehabilitative efforts, and diminish the need for joint replacement in this most common form of arthritis.

Disclosed herein are methods to treat osteoarthritis (OA), and/or to prevent the development of OA after a pre-disposing joint injury, by blocking or inhibiting the function of the molecule CD14. CD14 is a pattern-recognition receptor (PRR) of innate immunity and a co-receptor for several Toll-like Receptors (TLRs) and their ligands (damage-associated molecular patterns, or DAMPs), including TLRs and ligands that have been implicated in OA pathogenesis. CD14/TLR signaling promotes macrophage pro-inflammatory (M1) differentiation, modulates bone remodeling and pain. As existing treatments for OA are mainly short-acting analgesics, have no effect on progression of disease, and are fraught with significant side effects that limit their use, there is a need for new treatment approaches.

### Methods of Treatment

Disclosed herein are methods of treating osteoarthritis and/or preventing the development of osteoarthritis after a joint injury in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteoarthritis or preventing the development of osteoarthritis after a joint injury in the subject. In some aspects, the subject has osteoarthritis or can be at risk for developing osteoarthritis after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. For example, disclosed are methods of treating osteoarthritis or preventing the development of osteoarthritis after a joint injury in a subject, the method comprising: administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteoarthritis or preventing the development of osteoarthritis after a joint injury in the subject. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject.

Disclosed herein are methods of reducing or ameliorating one or more symptoms of osteoarthritis in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing or ameliorating one or more symptoms after a joint injury in the subject. In some aspects, the subject has osteoarthritis or can be at risk for developing osteoarthritis after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects, the one or more symptoms of osteoarthritis can be pain, pain-related functional decline, joint-related disability, pain sensitization, hyperalgesia, mechanical allodynia, cartilage degeneration, bone remodeling, synovitis, joint effusions, bone marrow lesions, subchondral cysts or a combination thereof. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed are methods of reducing or ameliorating one or more symptoms of osteoarthritis in a subject, the method comprising: administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing or ameliorating one or more symptoms of osteoarthritis in the subject.

Disclosed herein are methods of reducing inflammation in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing inflammation or reducing the development of inflammation after a joint injury in the subject. In some aspects, the subject has osteoarthritis or can be at risk for developing osteoarthritis after a joint injury. In some aspects, the subject has inflammation or can be at risk for developing inflammation after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed are methods of reducing inflammation in a subject, the method comprising: administering to the subject with osteoarthritis or at risk for developing osteoarthritis after a joint injury, a therapeutically effective amount of a CD14 inhibitor, wherein the therapeutically effective of the CD14 inhibitor amount neutralizes or blocks CD14, inhibits CD14 function, inhibits CD14 production, or a combination thereof, thereby reducing inflammation in the subject.

Disclosed herein are methods of reducing cartilage degradation in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing cartilage degradation or preventing the development of cartilage degradation after a joint injury in the subject. In some aspects, the subject has osteoarthritis or can be at risk for developing osteoarthritis after a joint injury. In some aspects, the subject has cartilage degradation or can be at risk for developing cartilage degradation after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed are methods of reducing cartilage degradation in a subject, the method comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing cartilage degradation in the subject.

Disclosed herein are methods of treating or preventing osteophytosis in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteophytosis or preventing the development of osteophytosis after a joint injury in the subject. In some aspects, the subject has osteoarthritis or can be at risk for developing osteoarthritis after a joint injury. In some aspects, the subject has osteophytosis or can be at risk for developing osteophytosis after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed are methods of treating or preventing osteophytosis in a subject, the method comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing osteophytosis in a subject.

Disclosed herein are methods of treating or preventing bone marrow lesions in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating bone marrow lesions or preventing the development of bone marrow lesions after a joint injury in the subject. In some aspects, the subject has osteoarthritis or can be at risk for developing osteoarthritis after a joint injury. In some aspects, the subject has bone marrow lesions or can be at risk for developing bone marrow lesions after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed herein are methods of treating or preventing bone marrow lesions in a subject, the method comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing bone marrow lesions in the subject.

Disclosed herein are methods of reducing or preventing bone pathology in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing bone pathology or preventing the development of bone pathology after a joint injury in the subject. In some aspects, the subject has osteoarthritis or can be at risk for developing bone pathology after a joint injury. In some aspects, the bone pathology can be or includes subchondral bone sclerosis, osteophytosis, one or more bone marrow lesions or a combination thereof. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed herein are methods of reducing or preventing bone pathology in a subject the method comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing or preventing bone pathology in the subject.

Disclosed herein are methods of treating or preventing subchondral bone sclerosis in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating subchondral bone sclerosis or preventing the development of subchondral bone sclerosis after a joint injury in the subject. In some aspects, the subject has osteoarthritis or can be at risk for developing subchondral bone sclerosis after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed are methods of treating or preventing subchondral bone sclerosis in a subject, the method comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing subchondral bone sclerosis in the subject.

Disclosed herein are methods of treating or preventing subchondral bone remodeling in a subject. In some aspects, the methods can comprise administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating subchondral bone remodeling or preventing the development of subchondral bone remodeling after a joint injury in the subject. In some aspects, the subject has osteoarthritis or can be at risk for developing subchondral bone remodeling after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject. For example, disclosed are methods of treating or preventing subchondral bone remodeling in a subject, the method comprising: administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing subchondral bone remodeling in the subject.

In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor.

In some aspects of the disclosed methods, the administration of the therapeutically effective amount of the CD14 inhibitor reduces fibrosis in the subject without altering the synovial hyperplasia or cellularity in the subject.

In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject.

In some aspects of the disclosed methods, the one or more symptoms of osteoarthritis can be pain. Examples of symptoms of osteoarthritis include but are not limited to joint stiffness, decreased range of motion (flexibility), swelling, pain-related functional decline, joint-related disability, pain sensitization, hyperalgesia, mechanical allodynia, cartilage degeneration, bone remodeling, synovitis, joint effusions, bone marrow lesions, and subchondral cysts.

In some aspects of the disclosed methods, the CD14 inhibitor can be Atibuclimab, recombinant thrombomodulin (rTMD23), microglial healing peptide 1 with N-terminal acetylation and C-terminal amidation (MHP1-AcN), iMAP2K3, or a combination thereof. In some aspects, the CD14 inhibitor can be an anti-CD14 antibody.

The compositions described herein can be formulated to include a therapeutically effective amount of one or more of the CD14 inhibitors described herein. Therapeutic administration encompasses prophylactic applications (e.g., or preventing the development of osteoarthritis after a joint injury or preventing subchondral bone sclerosis). Based on genetic testing and other prognostic methods, a physician in consultation with their patient can choose a prophylactic administration where the patient has a clinically determined predisposition or increased susceptibility (in some cases, a greatly increased susceptibility) to a osteoarthritis, cartilage degradation, or subchondral bone sclerosis.

The compositions (e.g., CD14 inhibitors) described herein can be administered to the subject (e.g., a human patient) in an amount sufficient to delay, reduce, or preferably prevent the onset of clinical disease. Accordingly, in some aspects, the patient can be a human patient. In some aspects, the subject can be a dog, a cat, a horse, a goat, or a non-human primate. In therapeutic applications, compositions can be administered to a subject (e.g., a human patient) already with or diagnosed with osteoarthritis, cartilage degradation, inflammation, or subchondral bone sclerosis or one or more symptoms of osteoarthritis or subchondral bone sclerosis in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder, condition or injury is delayed, hindered, or prevented, or the disease, disorder, condition or injury or a symptom of the disease, disorder, condition or injury is ameliorated or its frequency can be reduced. One or more of the symptoms can be less severe. Recovery can be accelerated in an individual who has been treated. For example, treatment of osteoarthritis or subchondral bone sclerosis may involve, for example, a reduction inflammation, a reduction in cartilage degradation, or a reduction or prevention of pain.

Osteoarthritis occurs when the protective cartilage that cushions the ends of bones wears down over time. In some aspects, osteoarthritis can damage or effect any joint. In some aspects, the joint can be in the hands, knees, hips, spine, feet, neck or shoulder.

Subchondral bone sclerosis is the hardening of the bone just below the cartilage surface, and often appears in the later stages of osteoarthritis. In some aspects, subchondral bone sclerosis can be found at the load-bearing joints. In some aspects, subchondral bone sclerosis can be found in the knees, hips, hands, feet or spine.

Disclosed herein, are methods of treating a patient with arthritis or preventing the degeneration of cartilage associated with arthritis or subchondral bone sclerosis. Disclosed are methods of treating a patient with arthritis or preventing the degeneration of cartilage associated with arthritis or subchondral bone sclerosis comprising administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteoarthritis or preventing the development of osteoarthritis after a joint injury in the subject. In some aspects, the subject has osteoarthritis or can be at risk for developing osteoarthritis after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects, the arthritis can be osteoarthritis. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject.

Also disclosed herein, are methods of treating or preventing subchondral bone sclerosis in a subject. Disclosed herein, are methods of treating or preventing subchondral bone sclerosis in a subject comprising administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteoarthritis or preventing the development of osteoarthritis after a joint injury in the subject. In some aspects, the subject has osteoarthritis or can be at risk for developing osteoarthritis after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects, the subject has been diagnosed with arthritis, osteoarthritis, or subchondral bone sclerosis prior to the administering step. In some aspects of the disclosed methods, the concentration of serum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject.

Further disclosed herein, are methods of treating or preventing subchondral bone remodeling in a subject. Disclosed herein, are methods of treating or preventing subchondral bone remodeling in a subject comprising administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteoarthritis or preventing the development of osteoarthritis after a joint injury in the subject. In some aspects, the subject has osteoarthritis or can be at risk for developing osteoarthritis after a joint injury. In some aspects, the CD14 inhibitor can be Atibuclimab, recombinant thrombomodulin (rTMD23), microglial healing peptide 1 with N-terminal acetylation and C-terminal amidation (MHP1-AcN), or iMAP2K3. In some aspects, the CD14 inhibitor can be an anti-CD14 antibody. In some aspects, one or more CD14 inhibitors can be administered to a subject in need thereof. In some aspects, the subject can be identified as being in need of treatment before the administration step. In some aspects, the subject has been diagnosed with arthritis, osteoarthritis, or subchondral bone remodeling prior to the administering step. In some aspects of the disclosed methods, the concentration of scrum cytokines are not altered after administration of the CD14 inhibitor. In some aspects of the disclosed methods, the concentration of RANK 1, CCL12, Leptin, TNF-alpha, and/or IL-10 is not altered after administration of the CD14 inhibitor. In some aspects of the disclosed methods, the concentration of serum sCD14 is not altered after administration of the CD14 inhibitor. In some aspects of the disclosed methods, the administration of the therapeutically effective amount of the CD14 inhibitor reduces fibrosis in the subject without altering the synovial hyperplasia or cellularity in the subject. In some aspects of the disclosed methods, the administration of the therapeutically effective amount of the CD14 inhibitor preserves or improves preservation of articular cartilage in the subject. In the some aspects, the articular cartilage can be in the femur of the subject. In the some aspects, the articular cartilage can be in the tibia of the subject.

The compositions described herein used in the disclosed methods can be formulated to include a therapeutically effective amount of the CD14 inhibitor disclosed herein. In some aspects, CD14 inhibitor thereof disclosed herein can be contained within a pharmaceutical formulation. In some aspects, the pharmaceutical formulation can be a unit dosage formulation.

The therapeutically effective amount or dosage of any of the CD14 inhibitors used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the symptoms), the age and physical characteristics of the subject and other considerations known to those of ordinary skill in the art. Dosages can be established using clinical approaches known to one of ordinary skill in the art. A therapeutically effective dosage of CD14 inhibitor can result in a decrease in severity of one or more disease symptoms, an increase in frequency and duration of disease symptom-free periods, or a prevention of impairment or disability due to the disease affliction. As disclosed therein, in some aspects a therapeutically effective amount of a CD14 inhibitor can decrease cartilage degradation, decrease synovial inflammation, or otherwise ameliorate symptoms in a subject without altering the concentrations of serum cytokines, RANK 1, CCL12, Leptin, TNF-alpha, IL-10, and/or serum sCD14.

The duration of treatment with any composition in the methods disclosed herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the CD14 inhibitor as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

### Pharmaceutical Compositions

As disclosed herein, are pharmaceutical compositions, comprising one or more of the therapeutic compositions or CD14 inhibitors disclosed herein. As disclosed herein, are pharmaceutical compositions, comprising a CD14 inhibitor and a pharmaceutical acceptable carrier described herein. In some aspects, the CD14 inhibitor can be formulated for oral or parental administration. In some aspects, the parental administration is intravenous, subcutaneous, intramuscular or direct injection. In some aspects, the CD14 inhibitor can be administered intramuscularly, intravenously, subcutaneously, orally, topically, transdermally, sublingually, or intra-articularly. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes any of the disclosed CD14 inhibitors dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

### Articles of Manufacture

The composition described herein can be packaged in a suitable container labeled, for example, for use as a therapy to treating osteoarthritis or preventing the development of osteoarthritis after a joint injury or any of the methods disclosed herein. Accordingly, packaged products (e.g., sterile containers containing the composition described herein and packaged for storage, shipment, or sale at concentrated or ready-to-use concentrations) and kits, including at least one or more of the CD14 inhibitor as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compositions can be provided in a concentrated form with a diluent and instructions for dilution.

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. CD14 Deficiency and Intra-Articular Injection with a CD14 Blocking Antibody Reduces Functional Decline and Prevents Cartilage Degeneration in a Mouse Model of Post-Traumatic Osteoarthritis

Targeting CD14, a co-receptor that facilitates Toll-like receptor (TLR) signaling, has advantages over existing anti-inflammatory agents. First, CD14 acts by targeting the pathways that initiate inflammation in response to joint damage, thus acting far upstream of available agents. By targeting the pathways that initiate inflammation in the joint, the effects of treatment will be more significant and sustained than existing therapies. The approach disclosed herein will be safer than existing therapies, as clinical studies with an anti-human CD14 blocking antibody, used for other indications, have suggested significant safety (Axtelle T, and Pribble J. J Endotoxin Res. 2003; 9(6):385-9).

For example, neutralizing CD14 can block osteoarthritis (OA) inflammation at its source. Many Toll-like Receptors (TLRs) and TLR ligands (DAMPs) are found in the OA joint. Binding of damage-associated molecular patterns (DAMPs) (i.e., S100 proteins, lipopolysaccharides (LPS), cartilage extracellular matrix (ECM) degradation products) to TLRs initiates cellular inflammation and production of cytokines and proteases that damage cartilage, and activates COX-2 to generate prostaglandins that drive pain. Soluble and membrane CD14 interact with several TLRs at the cell surface and inside the cell to augment DAMP-mediated inflammation. Blockade of CD14 will be more effective than current anti-inflammatory agents (e.g., corticosteroids or NSAIDs) used in OA, as this approach targets several TLR/DAMP interactions upstream of other agents.

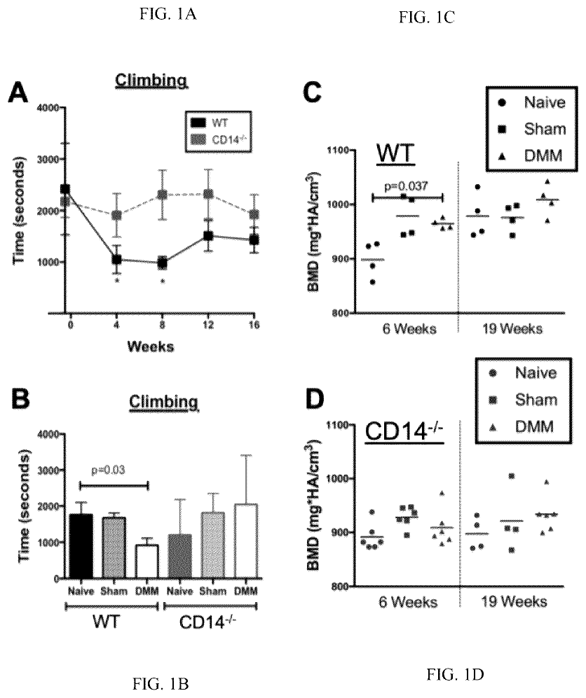

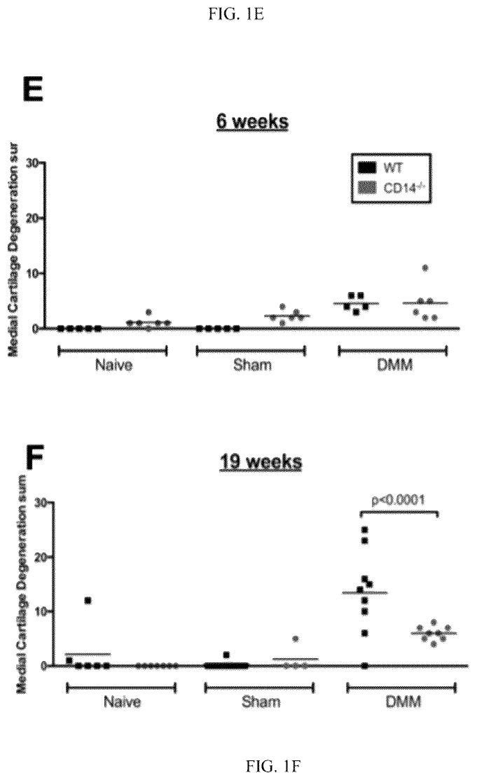

The results herein show that in mice deficient in the CD14 receptor (CD14−/− strain) and methods disclosed herein will be substantially more effective than existing treatments (Sambamurthy N, et al. PLOS One. 2018; 13 (11):e0206217). Using the destabilization of the medial meniscus model (DMM), CD14−/− mice were protected from development of functional decline after injury, as demonstrated by preserved climbing activity after injury (FIGS. 1A-B). Reductions in climbing activity after DMM injury in the wild type strain have been demonstrated to be related to joint pain in this model (Inglis J J, et al. Arthritis Rheum. 2008; 58(10):3110-9; and Miller R E, et al. Proceedings of the National Academy of Sciences of the United States of America. 2012; 109 (50):20602-7). This model exhibits typical OA joint pathology after injury, including cartilage degeneration and bone remodeling. In addition to protection from function decline, the CD14 deficient mice showed reduced progression of cartilage degeneration after injury as well as reduced bone remodeling response (FIGS. 1C-F). NSAIDs and glucocorticoids, though effective in blocking OA pain temporarily in some patients, have not been demonstrated to have any substantial effect on progression of structural damage in mouse models, while deficiency of CD14 improved pain-related outcomes and structural damage.

Methods and compositions to treat osteoarthritis via blocking or inhibiting CD14 function pharmacologically are described in Table 1. For example, monoclonal antibody blockade or small molecule inhibition approaches can be used.

TABLE 1

| Molecular | | |
|---|---|---|
| Target | Method | Delivery |
| Soluble and/ | Neutralizing/blocking | Systemic (i.e., intravenous, |
| or membrane | antibody (Axtelle T, et al. J | subcutaneous, intramuscular) |
| CD14 | Endotoxin Res. | or local (intra-articular) |
| | 2003; 9(6):385-9) or | |
| | fragment thereof | |
| Soluble and/ | Small molecule or peptide | Systemic (i.e., intravenous, |
| or membrane | inhibitor of CD14 function | subcutaneous, intramuscular, |
| CD14 | (Ma CY, et al. J Immunol. | oral) or local (intra-articular) |
| | 2015; 194(4): 1905-15; | |
| | and Ju N, et al. | |
| | ImmunoHorizons | |
| | 2021, 5 (6): 438-447) | |
| Soluble and/ | Small molecule inhibitor of | Systemic (i.e., intravenous, |
| or membrane | CD14 production (Jimenez- | subcutaneous, intramuscular, |
| CD14 | Duran G, et al. | oral) or local (intra-articular) |
| | eBioMedicine | |
| | 2020, 61: 103039) | |

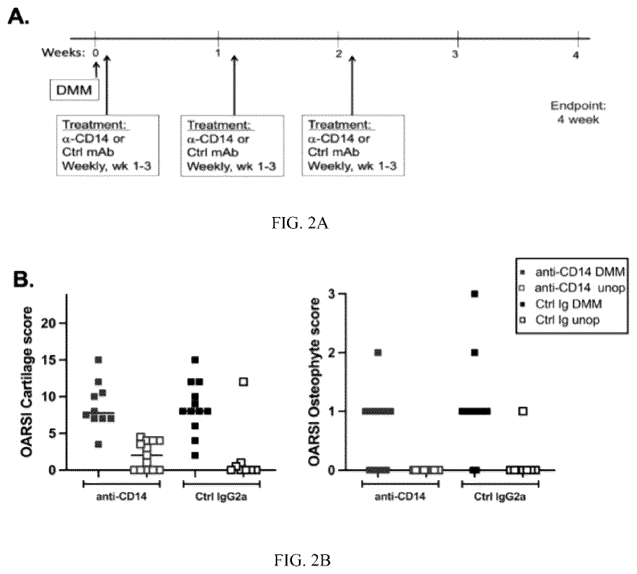

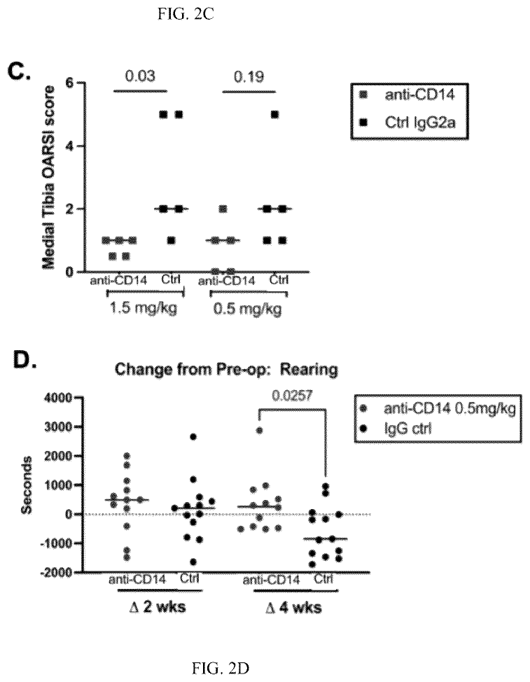

The effects of a CD14 neutralizing monoclonal antibody (mAb) was tested by, injecting intra-articularly (IA—into the knee joint) after DMM injury, in mice. As some anti-inflammatory treatments (e.g., glucocorticoids) have demonstrated deleterious effects on joint tissues, a short-term (4 week) safety study was conducted to ensure that IA CD14 blockade would not worsen joint pathology. DMM-operated C57BL/6 male mice were treated with a neutralizing anti-murine CD14 antibody (clone biG 53, Cell Sciences Inc.), or an isotype-matched (IgG2a) antibody delivered IA by knee injection, administering 0.5 mg/kg in three weekly doses starting 2 days after DMM surgery (FIG. 2A). LABORAS outcomes were measured at 2 and 4 weeks post-DMM, and histopathologic analysis of cartilage degeneration and osteophytosis (bone spur formation) was performed on knees harvested at 4 weeks per established protocols13. No worsening of early cartilage damage or early osteophyte development was observed with treatment (FIG. 2B), demonstrating the safety of this approach, and comparison with a higher dose (1.5 mg/kg) showed protective effects on medial cartilage pathology (FIG. 2C). When evaluating function decline after knee injury, decline in spontaneous activity (specifically rearing activity) was observed 4 weeks after DMM surgery in control treated mice. The decline in rearing activity after injury was prevented by anti-CD14 treatment (0.5 mg/kg, FIG. 2D) demonstrating a therapeutic effect. An anti-mouse CD14 antibody (clone biG 53 purchased from Cellsciences (Newburyport, MA)) was used in these experiments.

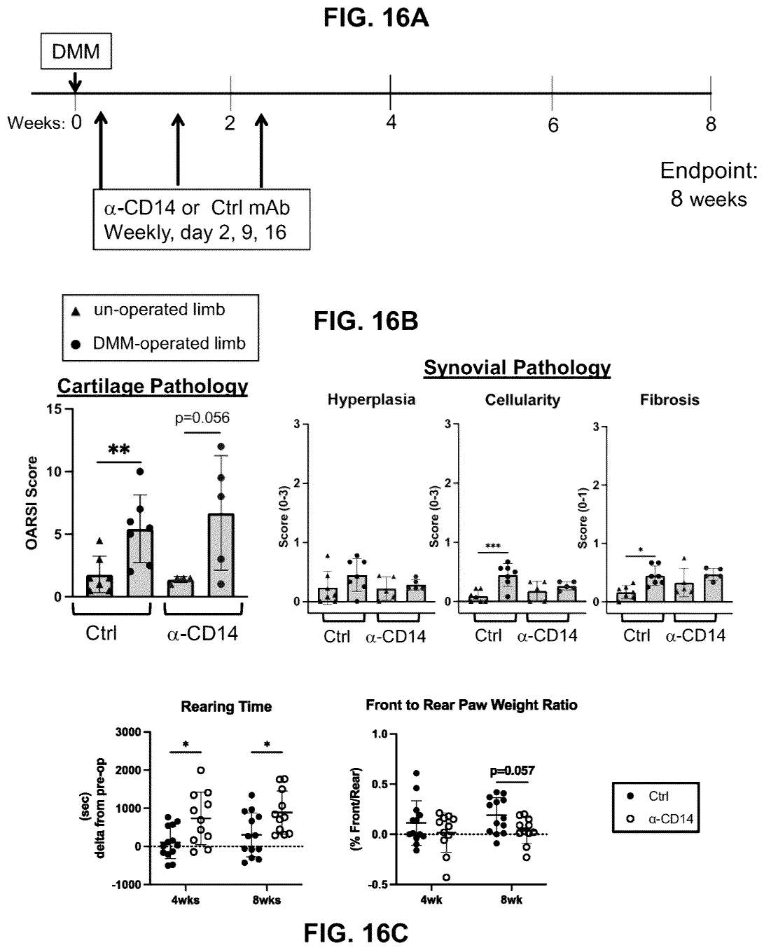

FIGS. 16B-C show the effects of intra-articular (IA) injection of a CD14 blocking antibody on pain and structural outcomes in the DMM model of post-traumatic osteoarthritis (PTOA) tested with an 8-week endpoint. FIG. 16A is a schematic of the experimental design. Twelve-week-old male C57BL/6 mice were subjected to DMM-knee injury on the right hindlimb. Starting 2 days after surgery, two groups of mice were then treated weekly for 3 weeks with intra-articular injection of either the anti-CD14 or a control monoclonal antibody (mAb) at 0.5 mg/kg. The results show that 8-wks post DMM, no significant difference was observed in cartilage pathology scoring (OARSI score) in DMM-operated knees between anti-CD14 and control treated groups. No significant differences in synovial pathology (lining hyperplasia, cellularity and fibrosis) was observed in CD14-treated knees compared with IgG-controls, but differences in synovial cellularity and fibrosis in DMM-operated knees compared to unoperated knees were observed only in the control-treated group (cellularity p<0.0001; fibrosis p=0.0078) (FIG. 16B). At 4- and 8 weeks-post-DMM, evaluation of spontaneous cage behaviors was performed using the Laboratory Animal Behavior Observation Registration and Analysis System (LABORAS™, Metris). FIG. 16C also shows that anti-CD14 treatment significantly increased rearing time at 4- and 8-wks post DMM, compared to control treated mice (p<0.05). Additionally, paw weight bearing distribution was measured via the Advanced Dynamic Weight Bearing (ADWB, Bioseb) system. A decreasing trend (p=0.057) in weight shifting from the rear to the front paws (front to rear paw weight ratio) was also observed at 8-weeks post DMM in the anti-CD14 treated mice compared to controls.

### Example 2. Anti-CD14 Treatment Reduces Mechanical Allodynia and Other Pain-Related Behaviors in Two Mouse Models of Post-Traumatic Osteoarthritis

Serum sCD14 levels and pain behavior were increased with single load knee compression injury that ruptured the anterior cruciate ligament compared to sham at 4 weeks post injury in female mice.

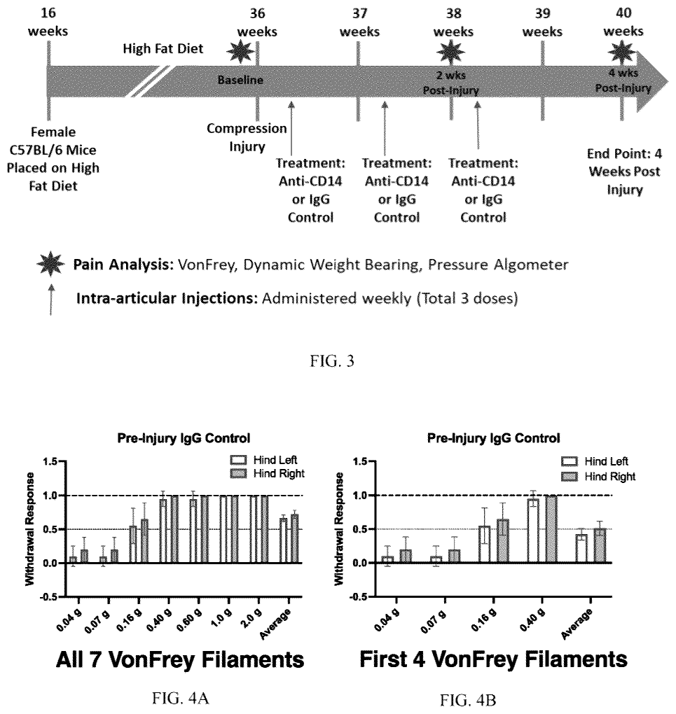

The effects of an anti-CD14 antibody on pain and structural outcomes in a single load knee compression injury model of post-traumatic osteoarthritis (PTOA) was tested. FIG. 3 shows the design of the experiment. The following outcomes will be measured: pain, inflammation, and structural pathology (including histology and imaging mass cytometry).

Von Frey, dynamic weight bearing and algometer will be used to measure pain-related behaviors. Von Frey is used to measure an evoked behavioral response. Von Frey Filaments are applied in ascending order to left and right hind paws, repeated twice; filaments (force): 0.04 g, 0.07 g, 0.16 g, 0.4 g, 0.6 g, 1 g, and 2 g. Scoring: positive response (1); or negative response (0) are recorded.

Dynamic weight bearing is used to measure spontaneous behavioral responses. For this, mice are placed in the chamber and allowed to freely move about for 5 minutes. Auto-scored “level one” data (highest confidence) were exported and analyzed. Outcome: magnitude and duration of body weight supported by each limb.

An algometer is used measure evoked behavioral responses. Pressure was applied to the knee joint using the algometer until the mouse responds or the maximum threshold (1000 g) is reached. The withdrawal force (or maximum force) was recorded for each knee joint following 3 rounds of testing.

Weekly weight measurements. 16-Week-old C57BL/6 mice were place on a high fat diet until the end of the study at 40 weeks of age.

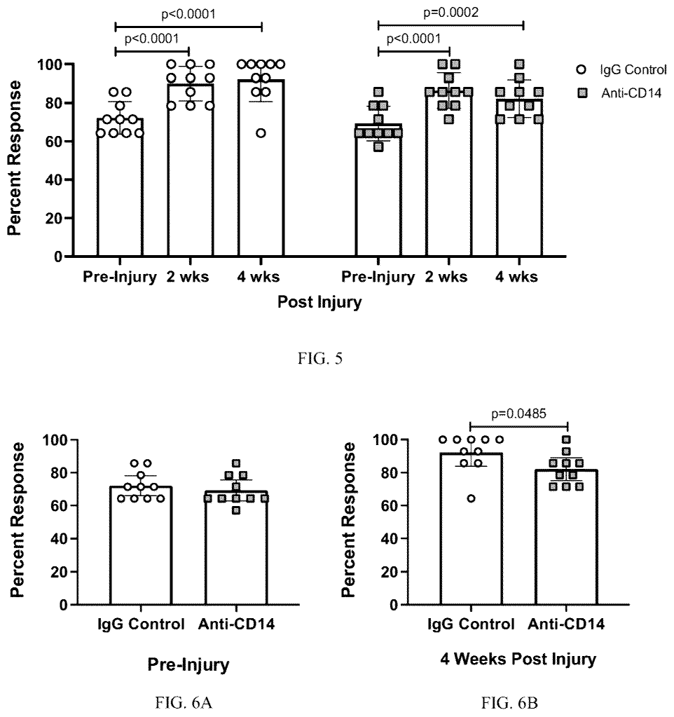

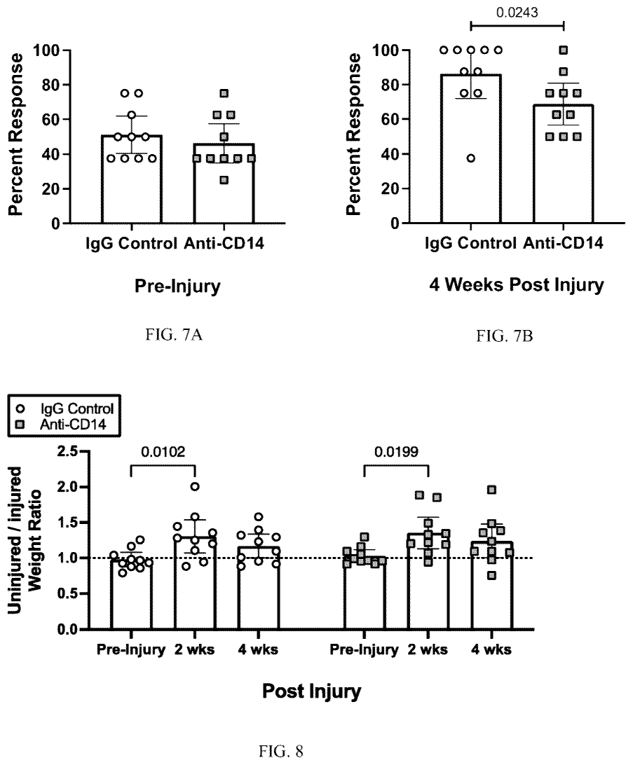

Evoked behavioral responses results. FIGS. 4A-B show the pre-injury von Frey filament responses. FIG. 5 shows the effects of injury on mechanical allodynia (increased percent response to the filaments from pre-injury baseline) in mice 4 weeks post compression injury measured by von Frey. FIGS. 6A-B show that compared to IgG control, anti-CD14 treatment significantly decreased mechanical allodynia by approximately one-third at 4 weeks following injury. FIGS. 7A-B show that compared to IgG control, anti-CD14 treatment significantly decreased mechanical allodynia by approximately one-third at 4 weeks following injury. The pre-injury averages with all seven filaments were higher than expected. Average from all seven filaments: pre-injury, control: 72.1%; and anti-CD14: 69.3%; and 4 weeks post injury, control: 92.1%; and anti-CD14: 82.1%. Average from first four filaments: pre-injury, control: 51.3%; and anti-CD14: 46.3%; and 4 weeks post injury, control: 86.3%; and anti-CD14: 68.8%.

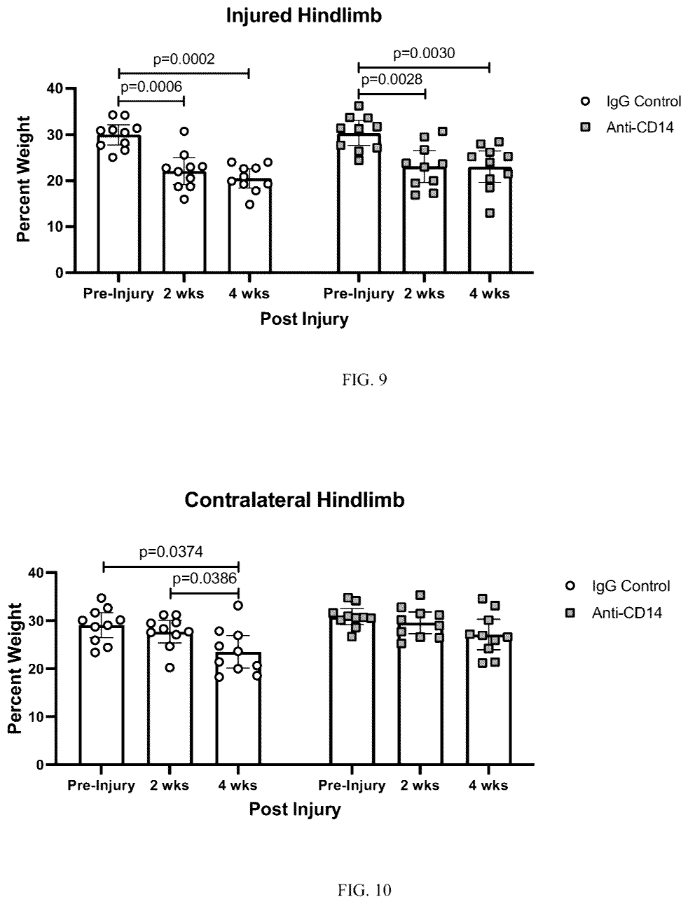

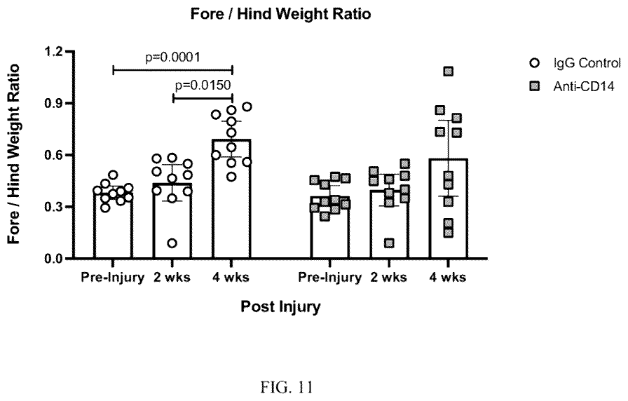

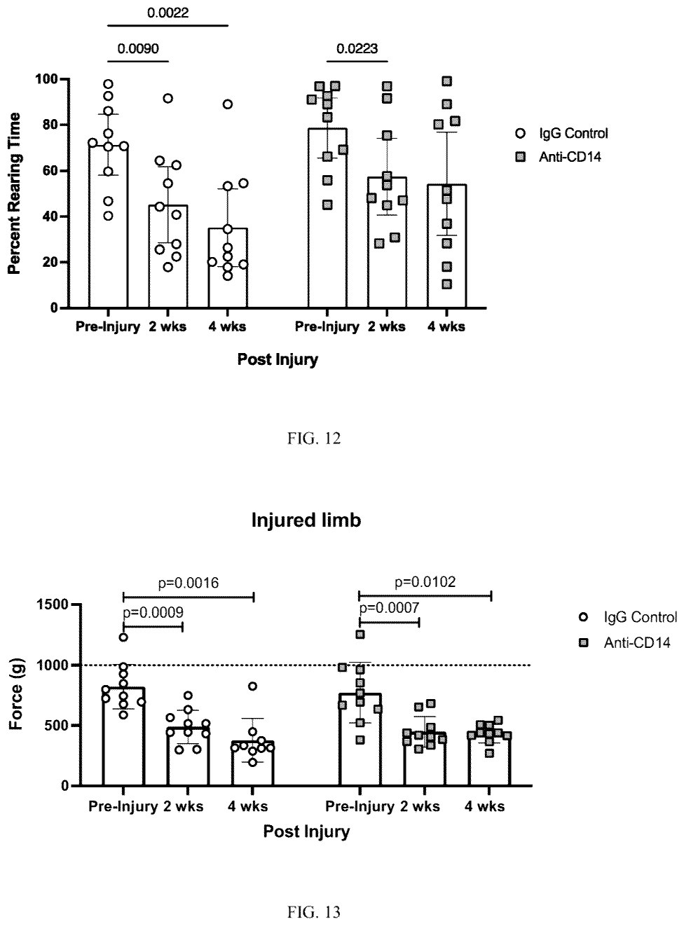

Spontaneous behavioral response results. FIG. 8 shows that mice developed pain sensitization 2 weeks post compression injury. Ratio>1=Less body weight support on the injured limb. FIG. 9 shows the percent weight support in the hindlimb decreased from pre-injury to 4 weeks post compression injury in the control and anti-CD14 group injured limb. FIG. 10 shows that weight support decreased in the control group contralateral limb but not in the anti-CD14 group contralateral limb. FIG. 11 shows that the percent weight support in the forelimbs relative to hindlimbs increased from pre-injury to 4 weeks post compression injury in the control but not in the anti-CD14 group. Increase Ratio=More body weight support on the forelimbs relative to hindlimbs. FIG. 12 shows that the time mice spent rearing decreased at 2- and 4-weeks post compression injury in the control group and only at 2 weeks post injury in the anti-CD14 group. Increase in percent time=increased rearing; and a decrease in percent time=decreased rearing.

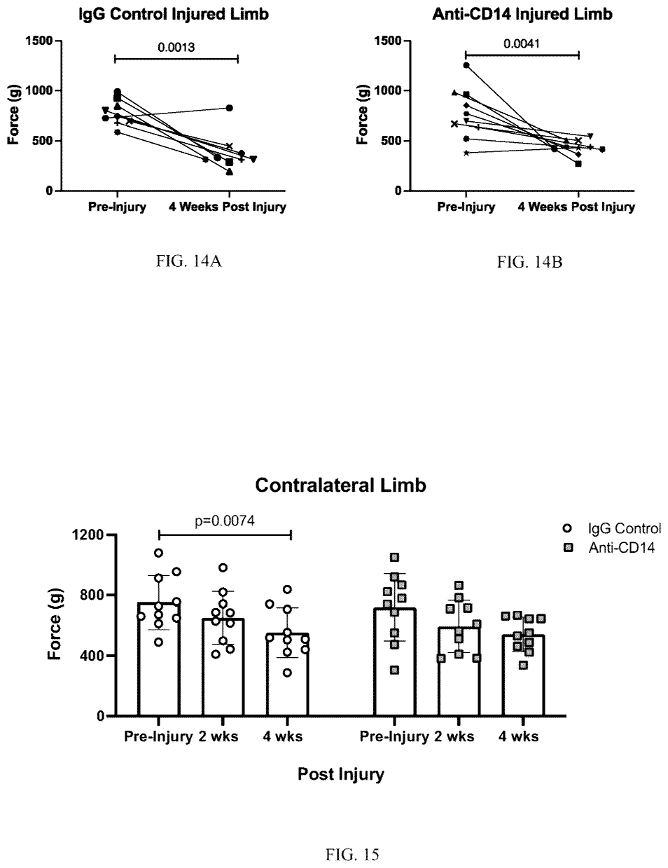

Evoked behavioral response results. FIG. 13 shows that the average force required to elicit a response decreased at 2- and 4-weeks post injury in the injured limb in both control and anti-CD14 groups. FIGS. 14A-B show the withdrawal force pre- and 4-weeks post injury using a paired analysis to illustrate the variation in individual responses for control and anti-CD14 antibody on pain behaviors. FIG. 15 shows that in the control group, the force required to elicit a response decreased at 4 weeks post injury in the contralateral limb but not in the anti-CD14 group. No difference was observed in the force required to elicit a response at 4 weeks post injury.

Serum cytokine analysis. Serum inflammatory markers (at 4 weeks post injury) were quantified according to the manufacturer's protocol using R&D (BioTechne) Quantikine ELISA Mouse CD14 and Multi-Analyte Profiling (xMAP) Luminex Assay. The analytes measured include: CCL2/JE/MCP-1 (BR18), CCL12/MCP-5 (BR42), IL-1 alpha/IL-1F1 (BR47), IL-1 beta/IL-1F2 (BR19), IL-6 (BR27), IL-10 (BR28), IL-17/IL-17A (BR30), Leptin/OB (BR35), beta-NGF (BR43), TNF-alpha (BR14), and TRANCE/TNFSF11/RANK L (BR25). The effects of injury and anti-CD14 on serum soluble CD14 (sCD14) levels in mice 4 weeks post compression injury were measured. Serum sCD14 was quantified by mouse CD14 Quantikine enzyme linked immunosorbent assay (R&D Systems). No difference in sCD14 levels was observed between the control and anti-CD14 group 4 weeks post-injury. Serum cytokines were not altered in anti-CD14 group 4 weeks post injury compared to control group for RANK L, CCL12, Leptin, TNF-alpha and IL-10.

Compared to the IgG control, anti-CD14 treatment significantly reduced mechanical allodynia by approximately one third at 4 weeks post injury group. The anti-CD14 group maintained rearing behavior which reduced the shift from the injured hindlimb to the forelimb compared to the IgG control group. Anti-CD14 treatment did not improve the pressure pain threshold and did not alter the levels of serum sCD14 and other serum cytokines compared to the IgG control. An anti-mouse CD14 antibody (clone biG 53 purchased from Cellsciences (Newburyport, MA)) was used in these experiments.

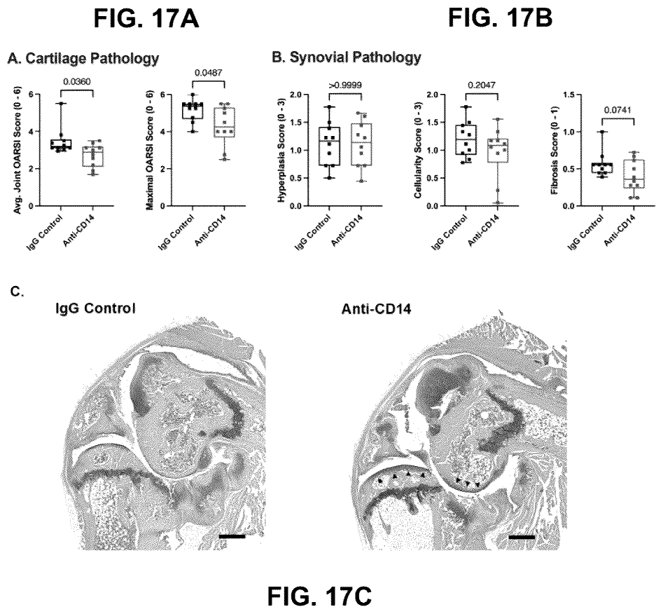

Anti-CD14 treatment also reduces knee joint pathology in loading-induced injury model of PTOA. Anterior cruciate ligament rupture (ACLR) was induced in 36-week-old adult female mice that were placed on a 45% kcal high fat diet beginning at 16 weeks of age. Anti-CD14 (5 mg/kg) or IgG control (5 mg/kg) treatments were administered by intra-articular injection 3 days after injury and weekly thereafter for a total of 3 treatments. FIG. 17A shows the results of an evaluation of anti-CD14 treatment versus IgG control on femoral and tibial cartilage pathology using the OARSI scoring system (range: 0-6) in the medial and lateral compartments by 2 experienced graders blinded to sample identification. Anti-CD14 treatment significantly reduced cartilage pathology based on two different analytic approaches: (i) average joint score, which is the average score of the evaluated sites (e.g., femur and tibia from the medial and lateral joint compartments), and (ii) maximal joint score, which uses the maximal score recorded from any site as the representative score for that joint. FIG. 17B shows that that anti-CD14 treatment reduced fibrosis (score averaged for all sites), but did not alter synovial hyperplasia or cellularity. FIG. 17C shows that anti-CD14 treatment improved preservation of articular cartilage along the femur and tibia.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating osteoarthritis or preventing the development of osteoarthritis after a joint injury in a subject, the method comprising:
administering to the subject in need thereof, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating osteoarthritis or preventing the development of osteoarthritis after a joint injury in the subject.

2. The method of claim 1, wherein the method reduces or ameliorates one or more symptoms of osteoarthritis in the subject.

3. (canceled)

4. A method of reducing cartilage degradation in a subject, the method comprising:
administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby reducing cartilage degradation in the subject.

5. A method of treating or preventing subchondral bone sclerosis in a subject, the method comprising:
administering to the subject, a therapeutically effective amount of a CD14 inhibitor, wherein the therapeutically effective amount of the CD14 inhibitor neutralizes or blocks CD14, inhibits CD14 function, inhibits CD14 production, or a combination thereof, thereby treating or preventing subchondral bone sclerosis in the subject.

6. The method of claim 4, wherein the subject has osteoarthritis or is at risk for developing osteoarthritis after a joint injury.

7. The method of claim 1, wherein the CD14 inhibitor is Atibuclimab, recombinant thrombomodulin (rTMD23), microglial healing peptide 1 with N-terminal acetylation and C-terminal amidation (MHP1-AcN), or iMAP2K3.

8. The method of claim 1, wherein the therapeutically effective amount of the CD14 inhibitor is administered intramuscularly, intravenously, subcutaneously, orally, topically, transdermally, sublingually, or intra-articularly.

9. The method of claim 1, wherein the subject is a human.

10. The method of claim 1, wherein the subject is a dog, a cat, a horse, a goat, or a non-human primate.

11. The method of claim 2, wherein the one or more symptoms of osteoarthritis is pain, pain-related functional decline, joint-related disability, pain sensitization, hyperalgesia, mechanical allodynia, cartilage degeneration, bone remodeling, synovitis, joint effusions, bone marrow lesions, or subchondral cysts.

12. The method of claim 1, wherein the subject is identified as being in need of treatment before the administration step.

13. The method of claim 1, wherein the concentration of serum cytokines are not altered.

14. The method of claim 1, wherein the concentration of RANK 1, CCL12, Leptin, TNF-alpha, and/or IL-10 is not altered.

15. The method of claim 1, wherein the concentration of serum sCD14 is not altered.

16. The method of claim 1, wherein the administration of the therapeutically effective amount of the CD14 inhibitor reduces fibrosis in the subject without altering the synovial hyperplasia or cellularity in the subject.

17. The method of claim 1, wherein the administration of the therapeutically effective amount of the CD14 inhibitor preserves articular cartilage in the subject.

18. The method of claim 1, wherein the administration of the therapeutically effective amount of a CD14 inhibitor preserves articular cartilage in the femur or tibia of the subject.

19. The method of claim 4, wherein the CD14 inhibitor is Atibuclimab, recombinant thrombomodulin (rTMD23), microglial healing peptide 1 with N-terminal acetylation and C-terminal amidation (MHP1-AcN), or iMAP2K3.

20. The method of claim 5, wherein the CD14 inhibitor is Atibuclimab, recombinant thrombomodulin (rTMD23), microglial healing peptide 1 with N-terminal acetylation and C-terminal amidation (MHP1-AcN), or iMAP2K3.
